(19) United States
(12) Patent Application Publication
(10) Pub. No.: US 2021/0379214 A1
(43) Pub. Date: Dec. 9, 2021

(54) Phospholipid-Free Small Unilamellar Vesicles (PFSUVS) for Drug Delivery

(71) Applicant: The University of British Columbia, Vancouver (CA)

(72) Inventors: Shyh-Dar Li, Vancouver (CA); Wunan Zhang, Vancouver (CA); Po-Han Chao, Vancouver (CA); Roland Boettger, Vancouver (CA)

(21) Appl. No.: 17/285,775

(22) PCT Filed: Oct. 31, 2019
(86) PCT No.: PCT/CA2019/051547

§ 371 Date: Apr. 15, 2021

Related U.S. Application Data
(60) Provisional application No. 62/754,072, filed on Nov. 1, 2018.

(51) Int. Cl. A61K 51/12; A61K 31/7048; A61K 31/4706; A61K 31/437; A61K 31/12; A61K 31/167; A61K 49/18; A61P 1/16; A61P 35/00; A61K 47/28; A61K 47/26; A61K 9/107

(52) U.S. Cl. CPC A61K 51/1224; A61K 31/7048; A61K 31/4706; A61K 31/437; A61K 31/12; A61K 9/1075; A61K 49/1809; A61P 1/16; A61P 35/00; A61K 47/28; A61K 47/26; A61K 31/167

(57) ABSTRACT

Provided herein are phospholipid-free small unilamellar vesicle (PFSUV) compositions, including a steroid and a non-ionic surfactant wherein the molar ratio of steroid:nonionic surfactant is between 3:1 to 5:1. Furthermore, the compositions described herein were found to useful for drug loading, drug delivery where preferential targeting of drugs to the liver is of interest.

### CROSS REFERENCE TO RELATED APPLICATION

This application claims the benefit of U.S. Provisional Patent Application Ser. No. 62/754,072 filed on 1 Nov. 2018, entitled “PHOSPHOLIPID-FREE SMALL UNILAMELLAR VESICLES FOR DRUG DELIVERY”.

### TECHNICAL FIELD

The present invention relates to the field of drug delivery. In particular, the invention relates to phospholipid-free small unilamellar vesicles, methods for making and methods and uses for drug delivery.

### BACKGROUND

Liposomes are vehicles composed of phospholipids and cholesterol, containing a bilayer structure that separates the inner aqueous core from the external phase. Liposomes are biodegradable and biocompatible with low toxicity and immunogenicity. Hydrophilic and lipophilic drugs can be both loaded into the aqueous core and the lipid bilayer, respectively. Liposomes are a versatile drug delivery system with several products approved clinically as reviewed in (Mallick and Choi 2014).

Bastiat et al. (Bastiat, Oliger et al. 2007) reported that palmitic acid and cholesterol could be used as lipid components to form phospholipid-free liposomal vesicles. Melted palmitic acid molecules provide a nonpolar environment for solubilizing cholesterol, which straightens the fatty acid chains, promoting a molecular order compatible with the bilayer formation (Paré and Lafleur 2001). Similarly, non-ionic surfactants alone or with cholesterol have an ability to form vesicles that could encapsulate hydrophilic compounds, suggesting the existence of a lipid bilayer in the system. These non-ionic surfactant containing vesicles are named as niosomes and have been studied for their application in drug delivery. Different types of non-ionic surfactants have been utilized in niosomal vehicle (Moghassemi and Hadjizadeh 2014). Tween, Span and Brij were three most commonly used surfactants in niosomes (Fang, Hong et al. 2001) (Manosroi, Khanrin et al. 2010) (Manconi, Valenti et al. 2003). Two parameters are considered for the optimal surfactant candidate; hydrophilic-lipophilic balance (HLB) and critical packing parameter (CPP). HLB is a parameter to describe the degree of hydrophilicity or lipophilicity of a surfactant. On a scale from 0 to 20, a larger HLB value indicates that the surfactant is more water soluble. Surfactants with a HLB number between 3 and 8 are able to form niosome by themselves (Moghassemi and Hadjizadeh 2014). Surfactants with higher HLB can also form a bilayer structure with the help of other materials by neutralizing the strong hydrophilicity.

The critical packing parameter (CPP) predicts the molecular self-assembly in surfactant solution (Nagarajan 2002). It is defined by the equation: CPP=v/l_{c} a₀, where v, l_{c} and a₀ refer to hydrophobic group volume, critical hydrophobic group length and the area of the hydrophilic head group, respectively. CPP can predict the general size and shape of the surfactant. The bilayer of the niosome can only form when the CPP parameter ranges from 0.5 to 1, beyond which the head groups of the surfactants are either too big or too small. Only in this circumstance can each surfactant molecule occupy a rectangular geometry instead of a conical shape, which is the cornerstone of the bilayer structure. HLB and CPP are regarded as important tools for surfactant screening in niosomal formulations. However, the estimation of these parameters is regarded as hypothetical rather than empirical and can only be used, in the case of a single component niosome (Khalil and Zarari 2014).

Cholesterol is another vital component of niosomes. By introducing a hydrophobic group into the niosomal membrane system, cholesterol enlarges the reservoir of surfactant candidate. It can affect the niosome's pharmaceutical parameters including morphology, encapsulation efficiency, stability and in vivo behavior. Cholesterol is known to react with surfactant molecules through hydrogen bonding (Lipshultz, Colan et al. 1991). Upon integration into the niosome, cholesterol is able to influence transition temperature of their lipid membrane. In a previous study, 30% cholesterol (molar ratio) in a cholesterol/Span system was sufficient to impart a residual gel/liquid transition enthalpy, a property known as thermo-responsiveness (Abdelkader, Ismail et al. 2010), whereas 50% cholesterol was capable of abolishing gel/liquid transition of the bilayer membranes, resulting in the loss of thermo-responsiveness (Abdelkader, Alani et al. 2014). A high ratio of cholesterol enhanced the vehicle's transition temperature and causes the niosome to stay in gel form at high temperature. Another parameter affected by cholesterol is the encapsulation efficiency (EE %). A span 20-based formulation has been reported that the increasing in cholesterol ratio resulted in a lowered EE % of timolol maleate from 45±2.3 to 30±1.5% (Abdelkader, Farghaly et al. 2014). Similar effects were also observed in Span 40 and Span 60 formulations (Abdelkader, Farghaly et al. 2014). However, some contradictory findings were also reported whereby increasing the cholesterol ratio can improve the EE % for a Span 85-based formulation (Abdelkader, Farghaly et al. 2014). The exact mechanism by which cholesterol can affect the encapsulation efficiency remains to be elucidated. One common finding from previous studies is that a 50% molar ratio of cholesterol is the optimal cholesterol percentage for the formulation stable niosome with high EE % (Mokhtar, Sammour et al. 2008).

Other lipids are also used as helper lipid with multiple functional purposes in niosomes, as an alternative to cholesterol. Cationic lipid is another helper lipid for niosomes used for gene delivery. Cationic lipid like N-[1-(2,3-dioleoyloxy)propyl]-N,N,N-trimethylammonium chloride (DOTAM) can interact with negatively charged DNA or RNA (Mashal, Attia et al. 2017), leading to the formation of a niosome-DNA or niosome RNA complex. Solulan C was used as a substitution of cholesterol and stabilized niosomes from aggregation (Yadav 2010). Dicetyl phosphate is another prevalent additive used to impart a negative charge on the niosomal surface to stabilize its bilayers (Waddad, Abbad et al. 2013). Helper lipids can also impact the endocytosis pathway of niosomes into a cell. Previously, Ediberto Ojeda et al. (Ojeda, Puras et al. 2016) reported that Tween 80™ niosomes incorporated with squalene had a 4-fold higher transfection efficiency into cells as compared to Tween 80/cholesterol niosomes due to a higher uptake and lysosomal escape. Also, Mohamed Mashal et al. (Mashal, Attia et al. 2017) have shown that the incorporation of lycopene into the Tween 60 niosome can not only enlarge niosomes' size from 66.49±1.17 nm to 101.6±2.48 nm, but also induce a higher transfection efficiency which is 10 times higher than in the absence of lycopene, potentially due to a pinocytosis and raft-mediated pathway of cellular uptake.

Niosomes have also been used for ocular delivery of therapeutic agents such as tacrolimus. (Li, Li et al. 2014). In some cases, hyaluronic acid coating of the niosome can facilitate ocular contact time of the formulation and drug bioavalibility (Zeng, Li et al. 2016). Other drugs like prednisolone (Gaafar), lomefloxacin HCl (Khalil, Abdelbary et al. 2017) were also used as model drugs to evaluate the potential application of niosome for ocular delivery. Niosomes have also been investigated for gene therapy by intravitreal and subretinal administration. A DOTMA/Tween 60 formulation showed increased in vitro transfection efficiency but also was able to transfect the outer segment of the retina (Mashal, Attia et al. 2017). Niosomal formulations have been widely used in transdermal therapy. Topical anti-inflammation therapy is one of the main applications for niosomal formulation. As examples, a Span 60 niosomal polyxamer gel indicated great potential for celecoxib delivery (Auda, Fathalla et al. 2016) and there have also been reports of benzoyl peroxide loaded niosomal gel formulations (Budhiraja and Dhingra 2015). Niosomal formulation of lacidipine have also been reported for use in hypertension therapy via transdermal delivery (Soliman, Abdelmalak et al. 2016).

Previous studies have also proposed the use of niosomes for anti-tumor therapy based on the stability and adjustable size of the particle. Niosomes composed of Span 60, cholesterol and choleth-24 can encapsulate DOX, an anthracycline anti-tumor reagent, in its hydrophilic core utilizing a passive loading strategy. This formulation indicated a longer blood retention time with AUC increased by 6 fold compared with free DOX. Tumor accumulation increased 1.5 fold in this study (Uchegbu 1995). Niosome formulations with metalloporphyrin complexes have also been described for use in cancer (Yuasa 2008).

Niosomes could be an attractive system for systemic delivery of drugs, if the size could be controlled below 200 nm with narrow size distribution, as then it would be possible to rely the enhanced permeation and retention (EPR) effect to increase the accumulation of the drug-loaded niosomes in the tumour. There remains a need for improved niosome formulations and formulation methods that will yield a smaller size distribution and a high ratio of hydrophilic surfactant on the surface to prevent the binding of serum proteins that lead to clearance.

### SUMMARY

The present invention is based in part, on the surprising discovery that phospholipid-free small unilamellar vehicle (PFSUV) particles with compositions of cholesterol:surfactant ranging between 1.5:1 and 5:1 (mol/mol) can be produced with a mean diameter of ≈80 nm, yet only the high-Cholesterol formulations (3:1 and 5:1) can retain a transmembrane gradient of ammonium sulfate for active loading of doxorubicin (DOX). Furthermore, it was surprisingly found that the PFSUV particles described herein are highly efficient and selective for hepatocyte cells for liver targeting. The inventors have for the first time manufactured a high cholesterol content (83% molar content) corresponding to a 5:1 mol/mol ratio of cholesterol:non-ionic surfactant using microfluidic methodology.

PFSUVs for these vesicles (phospholipid free small unilamellar vesicles) are meant to be distinguished from the more general category of niosomes due to their higher cholesterol content and smaller size.

Drugs may be loaded in the bilayer or the aqueous core of PFSUVs depending on the property of the drugs. For example, hydrophobic drugs (like, curcumin) can be loaded in the bilayer, while hydrophilic drugs are encapsulated in the core. For some compounds, they may be actively loaded into the core via a transmembrane gradient (e.g. DOX by the ammonium gradient). The drug loading principles that are known with liposomes also applies to PFSUVs.

The presently described PFSUV formulations designed for liver targeting of the drug or imaging agent. It is suspected that the reason that it is targeted to the liver, is that with the high cholesterol content, it then binds HDL/apoliproteins in the blood and then is trafficked to the liver and removed from circulation by the LDL receptor.

Accordingly, this would be a preferable way of formulating drugs or imaging agents where the disease site is the liver (for example, hepatitis, NASH, HCC, liver-stage malaria) and even more preferable when the disease site is the liver and the drug or imaging agent has toxic side effects outside of the liver.

In accordance with one embodiment, there is provided a phospholipid-free small unilamellar vesicle (PFSUV) composition, wherein the composition includes: (a) a steroid; and (b) a nonionic surfactant; wherein the molar ratio of steroid:nonionic surfactant may be between 3:1 to 5:1.

Alternatively, the molar ratio of steroid:nonionic surfactant may be between 4:1 to 5:1. Alternatively, the molar ratio of steroid:nonionic surfactant may be between 3:1 to 4:1.

In accordance with a further embodiment, there is provided a method for treating a liver disease, the method including administering an effective amount of a composition described herein to a subject in need thereof.

In accordance with a further embodiment, there is provided a composition described herein, for treating a liver disease.

In accordance with a further embodiment, there is provided a pharmaceutical composition for treating liver disease, including a composition described herein and a pharmaceutically acceptable carrier.

In accordance with a further embodiment, there is provided a use of a composition described herein, for treating a liver disease.

In accordance with a further embodiment, there is provided a use of a pharmaceutical composition described herein for treating a liver disease.

In accordance with a further embodiment, there is provided a use of a composition described herein, in the manufacture of a medicament for treating a liver disease.

In accordance with a further embodiment, there is provided a use of a composition described herein, for diagnosis or staging a liver disease.

In accordance with a further embodiment, there is provided a use of a composition described herein in the manufacture of an imaging agent for diagnosis or staging a liver disease.

The nonionic surfactant may be selected from the following: Tween-80™; Tween-85™; Tween-65™; Tween-60™; Tween-40™; Tween-20™; Span 60™; Span 80™; Span 85™; Span 65®; Span 40™; Span 20™; Pluronic F-88™; polysorbate 20; a Triton X-100™; Brij 78™; Brij 52™; Brij 30™; Brij 56™; Brij 58™; Brij 35™; Myrj 52™; sorbitan ester; a polyglycerol alkyl ether; a glucosyl dialkyl ether; and a polyoxyethylene alkyl ether. The nonionic surfactant may be selected from the following: Tween-80™; Tween-85™; Tween-65™; Tween-60™; Tween-40™; and Tween-20™. The nonionic surfactant may be selected from the following: Span 60™; Span 80™; Span 85™; Span 65™; Span 40™; and Span 20™. The nonionic surfactant may be selected from the following: Pluronic F-88™; polysorbate 20; and Triton X-100™. The nonionic surfactant may be selected from the following: Brij 78™; Brij 52™; Brij 30™; Brij 56™; Brij 58™; and Brij 35™. The nonionic surfactant may be selected from the following: Myrj 52™; sorbitan ester; a polyglycerol alkyl ether; a glucosyl dialkyl ether; and a polyoxyethylene alkyl ether. The nonionic surfactant may be selected from the following: Tween-80™; Tween-85™; Tween-65™; and Tween-60™. The nonionic surfactant may be selected from the following: Span 60™; Span 80™; Span 85™; and Span 65™. The nonionic surfactant may be selected from the following: Pluronic F-88™; and Triton X-100™. The nonionic surfactant may be selected from the following: Brij 78™; Brij 52™; Brij 56™; and Brij 58™. The nonionic surfactant may be selected from the following: Tween-80™; Tween-85™; Tween-65™; Span 80™; Span 85™; Span 65™; Pluronic F-88™; Triton X-100™; Brij 78™; Brij 56™; and Brij 58™.

The steroid may be a sterol. The sterol may be selected from one or more of: cholesterol; campesterol; sitosterol; stigmasterol; and ergosterol. The sterol may be selected from one or more of: cholesterol; campesterol; sitosterol; and ergosterol. The sterol may be selected from one or more of: cholesterol; campesterol; stigmasterol; and ergosterol. The sterol may be selected from one or more of: cholesterol; sitosterol; stigmasterol; and ergosterol. The sterol may be selected from one or more of: cholesterol; campesterol; sitosterol; and stigmasterol. The sterol may be cholesterol. The steroid may be cholesterol and the nonionic surfactant may be Tween-80™.

The mean diameter may be below 100 nm as measured using dynamic light scattering. The mean diameter may be between 10 nm and 100 nm as measured using dynamic light scattering. The mean diameter may be about 80 nm as measured using dynamic light scattering.

The PFSUV composition may, optionally, further include an apolipoprotein component. The apolipoprotein component may be selected from one or more of: Apo A-1; Apo A-2; Apo A-4; Apo A-V or Apo A5; Apo B48; Apo B100; Apo C-I; Apo C-II; Apo C-Ill; Apo C-IV; Apo D; Apo E; Apo H; and Apo L. The apolipoprotein component may be integrated into the PFSUV membrane or the apolipoprotein component may interact with the surface of the PFSUV.

The composition may further include a drug or imaging agent. The drug or imaging agent may be targeted to the liver. The weight ratio of drug:lipid may be about 1:4 to 1:40. The weight ratio of drug:lipid may be about 1:5 to 1:40. The weight ratio of imaging agent:lipid may be about 1:4 to 1:40. The weight ratio of imaging agent:lipid may be about 1:5 to 1:40. The weight ratio of drug:lipid may be about 1:4 to 1:25. The weight ratio of drug:lipid may be about 1:5 to 1:25. The weight ratio of imaging agent:lipid may be about 1:4 to 1:25. The weight ratio of imaging agent:lipid may be about 1:5 to 1:25. The drug or imaging agent may be selected from: doxorubicin; chloroquine; imiquimod; R848; curcumin; and sodium diatrizoate. The drug or imaging agent may be selected from: doxorubicin; chloroquine; imiquimod; R848; curcumin; and sodium diatrizoate. The drug may be selected from: doxorubicin; chloroquine; imiquimod; and R848. The drug or imaging agent may be selected from: curcumin; and sodium diatrizoate.

The liver disease may be selected from one or more of the following: viral hepatitis; non-viral hepatitis; cholestatic liver disease; non-alcoholic steatohepatitis (NASH); non-alcoholic fatty liver disease (NAFLD); primary biliary cholangitis; liver fibrosis; biliary atresia; hemochromatosis; Wilson's disease; alpha-1 antitrypsin deficiency; hyperoxaluria; oxalosis with liver cirrhosis; hepatitis B; and liver cancer. The liver cancer may be selected from: hepatocellular carcinoma; intrahepatic cholangiocarcinoma; and hepatoblastoma.

In accordance with a further embodiment, there is provided a method of diagnosis or staging a liver disease, the method including administering to a subject a composition described herein.

The composition may comprise a diagnostic probe, a contrast agent, a radioactive agent, a radioactive dye, a radiopharmaceutical, a PET or a MRI imaging agent. The method may further include CT, SPECT, ultrasound, PET or MRI imaging. The method may further include diagnosis or staging a liver disease.

In accordance with a further embodiment, there is provided a method wherein the method may include: combining a steroid and a nonionic surfactant using a microfluidic mixer, wherein the molar ratio of steroid:nonionic surfactant may be between 3:1 to 5:1.

The microfluidic mixer may be a staggered herringbone mixer (SHM). The microfluidic mixer may use a two-channel microfluidic injection system. The steroid may be dissolved in ethanol at a final concentration of 10 mg/ml and mixed with 120 mM ammonium sulfate (AS) solution at a flow ratio of 1/3 between ethanol and the aqueous phase. The steroid may be dissolved in ethanol and mixed with a citric acid loading gradient of 300 mM. The method may further include loading of a drug or an imaging agent at a weight ratio of drug:lipid or imaging agent:lipid of about 1:4 to 1:40. The method may further include dialyzing the composition against HEPES buffered saline. The method may further include measuring steroid concentration in PFSUVs after dialysis. The method may further include measuring PFSUV size using dynamic light scattering. The method may further include loading of a drug or an imaging agent at a weight ratio of drug:lipid or imaging agent:lipid of about 1:4 to 1:25. The imaging agent may be selected from one or more of: a diagnostic probe; a contrast agent; a radioactive agent; a radioactive dye; and a radiopharmaceutical.

The invention relates to formulation methods and niosome compositions that is a phospholipid-free small unilamellar vehicle (PFSUV) that are effective as drug delivery agents.

In one aspect of the invention, the PFSUV compositions comprises a cholesterol component and a non-ionic surfactant at a molar ratio from about 3:1 to 5:1 (cholesterol:nonionic surfactant).

Examples of the nonionic surfactant component of the PFSUV composition may include one or more of, but is not limited to: Tween-80™; Tween-85™; Tween-65™; Tween-60™; Tween-40™; Tween-20™; Span 60™; Span 80™; Span 85™; Span 65™; Span 40™; Span 20™; Pluronic F-88™; polysorbate 20; sorbitan esters; polyglycerol alkyl ethers; glucosyl dialkyl ethers; polyoxyethylene alkyl ethers; Triton X-100™; Brij 78™; Brij 52™; Brij 30™; Brij 56™; Brij 58™; Brij 35™; and Myrj 52™.

Examples of the cholesterol component of the PFSUV composition may include one or more of, but is not limited to: cholesterol; steroids; sterols; and other cholesterol derivatives or precursors.

The PFSUV composition may, optionally, further contain an apolipoprotein component, examples of the apolipoprotein component of the PFSUV composition may include, but is not limited to: Apo A-1; Apo A-2; Apo A-4; Apo A-V or Apo A5; Apo B48; Apo B100; Apo C-I; Apo C-II; Apo C-III; Apo C-IV; Apo D; Apo E; Apo H; and Apo L or either alone or in combination with one of more thereof.

In some aspects of the invention, the apolipoprotein component may be integrated into the PFSUV membrane or the apolipoprotein component may interact with the surface of the PFSUV particle via the association between the steroid (for example, cholesterol) or non-ionic surfactant (for example, Tween80) component and the apolipoprotein component.

In another aspect of the invention, the PFSUV composition encapsulates a drug component as a drug delivery vehicle. In some aspects of the invention, the drug to lipid ratio is a weight ratio from about 1:4 to 1:25 (Drug:lipid).

In another aspect of the invention, there is provided methods of formulation for the PFSUV compositions of the invention, which methods comprise the use of a microfluidic system to obtain a particle size of less than 200 nm.

In another aspect of the invention, a loading gradient is used to formulate a drug-loaded PFSUV composition. The loading gradient may include, but is not limited to, an ammonium sulfate gradient, a citric acid gradient, a manganese ion gradient, a copper ion gradient, a calcium ion gradient and the like.

In some aspects of the invention, the PFSUV compositions may be useful for the delivery of therapeutic agents to particular organs or sites in the body. In some aspects of the invention, the PSFUV compositions may be used for the delivery of therapeutic agents to the liver, spleen or brain. In other aspects of the invention, the PFSUV compositions may be useful for the delivery of therapeutic agents to a tumor site in the body. In another aspect of the invention, the PFSUV compositions may be useful for the delivery of an imaging agent or probe to a particular organ or site in the body.

### BRIEF DESCRIPTION OF THE DRAWINGS

FIG. 1 shows (A) the stability of empty PFSUVs containing an ammonium sulfate gradient stored at 4° C. in HBS comprising a range of ratios of Cholsterol:Tween 80™; and (B) Cryo-EM images of PFSUVs (5:1) indicated the formation of a small unilamellar vesicular structure.

FIG. 2 shows a schematic of the preparation method for PFSUVs loaded with doxorubicin.

FIG. 3 shows (A) the impact of loading temperature and lipid composition on drug encapsulation efficiency for PFSUVs containing an ammonium sulfate gradient; (B) the impact of loading temperature and lipid composition on the vesicle size for PFSUVs containing an ammonium sulfate gradient; (C) the impact of loading temperature and lipid composition on drug encapsulation efficiency for PFSUVs containing a citric acid gradient; (D) the impact of loading temperature and lipid composition on the vesicle size for PFSUVs containing a citric acid gradient. Data shown is the mean±standard deviation (n=3).

FIG. 4 shows (A) loading kinetics of doxorubicin into PFSUVs (5:1) containing an ammonium sulfate gradient at different temperatures; (B) loading kinetics of doxorubicin into PFSUVs (5:1) containing a citric acid gradient at different temperatures. Data shown is the mean±standard deviation (n=3).

FIG. 5 shows (A) the percentage Encapsulation Efficiency of drugs for PFSUVs (5:1) containing an ammonium sulfate gradient with varying Drug/Lipid ratios; (B) the percentage Encapsulation Efficiency of drugs for PFSUVs (5:1) containing a citric acid gradient. Data shown is the mean±standard deviation (n=3).

FIG. 6 shows hemolytic toxicity of PFSUVs loaded with doxorubicin at varying concentration of drug. Data shown is the mean±standard deviation (n=3).

FIG. 7 shows cryo-TEM images of empty PFSUVs (A); and PFSUVs loaded with doxorubicin (B), with the arrow in Panel A indicating the bilayer structure and the arrow in Panel B indicate crystalline doxorubicin. Scale bar represents 100 nm.

FIG. 8 shows drug retention in the pegylated liposomal doxorubicin (PLD) shown as squares and the PFSUV loaded with doxorubicin (PSFUVs-DOX) shown as circles, when incubated in 50% serum and HBS at 37° C. Data shown is the mean±standard deviation (n=3).

FIG. 9 shows EMT6 cell viability after treatment over 3 days with either free doxorubicin (DOX), PSUV loaded doxorubicin (PFSUVs-DOX) or pegylated liposomal doxorubicin (PLD), at varying concentrations of doxorubicin. Squares represent PLD, circles represent PFSUVs-DOX and triangle icons represent DOX.

FIG. 10 shows a graphic representation of cellular uptake measured in either the absence or presence of 10% FBS (FBS(−)) and FBS(+), respectively) or in the presence of varying concentrations of apolipoproteins; 5 ug/ml (labelled as Low Apo), 20 ug/ml (labelled as Middle Apo), 100 ug/ml (labelled as High Apo).

FIG. 11 shows the plasma concentration of doxorubicin post injection of either PSUV loaded doxorubicin (PFSUVs-DOX shown as circles) or pegylated liposomal doxorubicin (PLD shown as squares. Data shown is the mean±standard deviation (n=3).

FIG. 12 shows the biodistribution profile of (A) pegylated liposomal doxorubicin (PLD) and (B) PSUV loaded doxorubicin (PFSUVs-DOX). Data shown is the mean±standard deviation (n=3).

FIG. 13 shows the uptake of PSUV loaded doxorubicin (PFSUVs-DOX) by the liver. Confocal microscopy images of liver sections taken from mice 2 hr after intravenous treatment with PFSUVs-DOX or PLD. Panel A shows a graphic representation of the total number of DOX-positive hepatocytes and sinusoidal cells per microscopy images (n=3). Panel B shows the corresponding percentage of DOX-positives cells of each cell type. ****:P<0.0001.

### DETAILED DESCRIPTION OF THE INVENTION

### Definitions

As used herein, a steroid is a biologically active organic compound with four rings arranged in a steroid core structure. The core steroid structure has seventeen carbon atoms, bonded in four “fused” rings: three six-member carbon rings (rings A, B and C) and one five-member cyclopentane ring (the D ring).

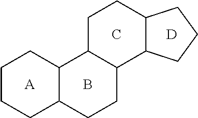

Steroids vary by the functional groups attached core rings and by the number of double bonds within the rings.

Steroids are important components of cell membranes and alter membrane fluidity and are found in plants, animals and fungi. Sterols are forms of steroids with a hydroxy group at position three and a skeleton derived from cholestane.

As used herein, a sterol or steroid alcohol, is a subgroup of the steroids and are a type of lipid. A common type of sterol is cholesterol, which is important for cell membrane structure.

Cholesterol has been shown to hydrogen bond to the hydrophilic head of some surfactants to improve the stability of the resulting niosome. Alternatively, sterols may be selected from cholesterol, campesterol, sitosterol, stigmasterol, ergosterol. The steroid may be a sterol. The sterol may be selected from one or more of: cholesterol; campesterol; sitosterol; stigmasterol; and ergosterol.

A nonionic surfactant may be selected from one or more of the following: Tween-80™; Tween-85™; Tween-65™; Tween-60™; Tween-40™; Tween-20™; Span 60™; Span 80™; Span 85™; Span 65™; Span 40™; Span 20™; Pluronic F-88™; polysorbate 20; a Triton X-100™; Brij 78™; Brij 52™; Brij 30™; Brij 56™; Brij 58™; Brij 35™; Myrj 52™; sorbitan ester; a polyglycerol alkyl ether; a glucosyl dialkyl ether; and a polyoxyethylene alkyl ether. The nonionic surfactant may be Tween-80™.

As used herein “an apolipoprotein” is a protein that bind lipids, including cholesterol, to form lipoproteins. Furthermore, apolipoproteins transport lipids, fat soluble vitamins in blood, cerebrospinal fluid and lymph. The apolipoprotein component may be selected from one or more of: Apo A-1; Apo A-2; Apo A-4; Apo A-V or Apo A5; Apo B48; Apo B100; Apo C-I; Apo C-II; Apo C-III; Apo C-IV; Apo D; Apo E; Apo H; and Apo L.

As used herein “a liver disease” refers to any disorder of the liver. For example, liver disease may be selected from one or more of the following: viral hepatitis; non-viral hepatitis; cholestatic liver disease; non-alcoholic steatohepatitis (NASH); non-alcoholic fatty liver disease (NAFLD); primary biliary cholangitis; liver fibrosis; biliary atresia; parasitic infections of the liver and genetic liver diseases such as hemochromatosis; Wilson's disease; alpha-1 antitrypsin deficiency; hyperoxaluria; oxalosis liver cirrhosis; hepatitis B; and liver cancer (for example, hepatocellular carcinoma, intrahepatic cholangiocarcinoma, and hepatoblastoma).

As used herein an “imaging agent” refers to any agent that provides more information about internal organs, cellular processes and tumors, as well as normal tissue and may be used to diagnose disease, stage a disease or monitor treatment effects. Imaging agents may also be referred to as diagnostic probes, contrast agents, radioactive agents, radioactive dyes or radiopharmaceuticals. Imaging may refer to MRI, PET, CT and x-ray, and may involve the use of an imaging agent. Imaging agents may be administered by mouth, enema, or injection into a vein, artery, or body cavity. The agents are typically absorbed by the body or passed out of the body in the urine or bowel movement.

For example, an MRI imaging agent is Gadolinium. PET and Nuclear Medicine imaging agents may be selected from one or more of: 64Cu-ATSM (64Cu diacetyl-bis(N4-methylthiosemicarbazone)); FDG: 18F-fluorodeoxyglucose (FDG); 18F-fluoride; FLT: 3′-deoxy-3′-[18F]fluorothymidine (FLT); FMISO; Gallium; Technetium-99m; and Thallium. For example, an x-ray imaging agent may be selected from one or more of: Barium; Gastrografin; and Iodine contrast agents.

Various alternative embodiments and examples are described herein. These embodiments and examples are illustrative and should not be construed as limiting the scope of the invention.

Materials and Methods

Reagents

Tween 80™, cholesterol, ammonium sulfate, sheep red blood cells, doxorubicin (DOX) and HEPES (4-(2-hydroxyethyl)-1-piperazineethanesulfonic acid) were purchase from Sigma-Aldrich™ (St. Louis, Mo.). Ultra-pure water was prepared in our laboratory using Milli-Q Synthesis System™ (Millipore™, Merck™, Darmstadt, Germany). Free cholesterol E assay kit was purchased from Wako Chemicals USA Inc. (Richmond, Va.). 1,2-Disteary-sn-glycero-3-phospatidylcholine (DSPC) and 1,2-disteroyl-sn-glycero-phosphatiylethanol-amine-N-[methoxy (polyethyleneglycol)-2000] (DSPE-PEG2000) were purchased from Avanti Polar Lipids™ (Alabaster, Ala.). Thiazolyl Blue tetrazolium bromide was purchased from Alfa Aesar™ (Tewksbury, Mass.). Fluoroshield with DAPI was purchased from Sigma™ (Laramine, Wyo.).

Preparation of PFSUVs

PFSUVs dispersed in 120 mM ammonium sulfate were produced by the NanoAssemblr Benchtop™. Fifty ml of the PFSUVs were subjected to a tangential flow filtration system (TFF™ system) (Ki2™, Kroso™, Spectrum Labs™, Canada) to remove ethanol, exchange the exterior phase to HBS and concentrate. In the TFF system, the PFSUVs flew through a diafiltration cartridge with a molecular weight cut-off of 50 kD (Midikros™, hollow fiber filter module, Spectrum Labs™, Canada) at a flow rate 140 ml/min. The PFSUVs were concentrated to 30 mg/ml.

PFSUVs with different Tween 80™/cholesterol molar ratios (1:1.5, 2:1, 3:1, 5:1, 8:1) were fabricated in a controlled nanoprecipitation process using a two-channel microfluidic system (NanoAssemblr™, Precision Nanosystems International™, Vancouver, BC, Canada). The NanoAssemblr was equipped with a microfluidic cartridge that contained the staggered herringbone mixer (SHM) design (dimensions 6.6×5.5×0.8 cm, Precision Nanosystems International™). Solutions were injected into the cartridge via polypropylene syringes (Becton, Dickinson and Company™, Franklin Lakes, N.J.) with a size of 10 and 3 mL for aqueous and organic phases, respectively. Lipids were dissolved in ethanol at a final concentration of 10 mg/ml and were mixed with 120 mM ammonium sulfate (AS) solution in the NanoAssemblr™ at a flow ratio of 1/3 between ethanol and the aqueous phase. In some studies, lipids dissolved in ethanol were mixed with citric acid (citric acid loading gradient of 300 mM) prior to entry in the NanoAssemblr™ and DOX loading (as described below). The total flow rate is 15 ml/min.

The mixture was then dialyzed (slide-A-Lyzer™, 10000 MWCO) against HEPES buffered saline (HBS, pH 7.4) for 12 h, with fresh HBS replaced at 2 h and 4 h. Cholesterol concentration in PFSUVs after dialysis was determined by a cholesterol E assay kit. Particle size was measured using a particle analyzer (Zetasizer NanoZS™, Malvern Instruments Ltd.™, Malven, UK).

Although the NanoAssemblr™ from Precision Nanosystems International™ was used to fabricate PFSUVs another staggered herringbone micromixer could be used (see DU, Y. et al. Biomicrofluidics (2010) 4(2): 024105; KWAK, T. J. et al. PLoS ONE (2016) 11(11): eo166068; STROOCK AD. Chaotic Mixer for Microchannels. Science (2002) 295: 647-651; HAMA, B. et al. Microfluidics and Nanofluidics (2018) 22(5): 1-14). Furthermore, alternative microfluidic micromixers are available and known to a person of skill in the art (CAI, G. et al. Micromachines (2007) 8:274).

Short-Term Storage Stability of Empty PFSUVs

Empty PFSUVs with different Tween 80/Cholesterol ratios were stored at 4° C. in a glass vial. At selected time points, the size of each sample was measured using dynamic light scattering (Zetasizer NanoZS™, Malvern Instruments Ltd.™, Malven, UK).

Doxorubicin (DOX) Loading

PFSUVs (2.0 mg total lipids) were incubated with 100 μg DOX in a total volume of 1 ml. The mixture was incubated for 1 h at 20° C., 37° C., 45° C. and 60° C., respectively and then quenched on ice for 2 min. Encapsulation efficiency (EE %) was calculated following a UV/Vis spectroscopy method described in an earlier publication with some modifications (Tagami, May et al. 2012). The method utilized the property of DOX whose maximum absorbance undertakes a red-shift from 480 nm to 600 nm when the pH increases to 14. Adding NaOH to PFSUVs increased the pH of the exterior buffer to 14 and the unencapsulated DOX revealed a maximum absorbance at 600 nm, while the loaded DOX exhibited little absorbance at 600 nm. Briefly, 10 μl of PFSUVs-Dox was mixed with 2 μl NaOH (4 M) and 2 μl HBS, and was then transferred immediately to a Thermo Scientific NanoDrop 2000™ spectrophotometer to detect the absorbance at 600 nm. The final encapsulation efficiency was calculated by the following equation.

$$EE \% = 1 - \frac{R s - R 0}{R 100 - R 0}$$

Where Rs is the absorbance of the sample. Ro is the absorbance of mixture containing 10 μl PFSUVs-DOX and 4 μl HBS. R100 is the absorbance of 10 μl PFSUV-DOX mixed with 2 μl NaOH (4M) and 2 μl Triton-X 100™ (10%).

Loading Kinetic

DOX (100 μg) was incubated with PFSUVs (1.5 mg total lipids) for 5, 15, 30 or 60 min at 20° C., 37° C. or 60° C. The mixture was quenched in an ice bath for 2 min to terminate the loading procedure. EE % was measured using the method described earlier.

Effect of Drug/Lipid Ratio

DOX and PFSUVs were mixed at different drug/lipid ratios (from 1:5 to 1:25) at 37° C. for 1 h, and the encapsulation efficiency was measured by the previous method.

Hemolysis Study

Forty μl sheep red blood cells (SRBC) were mixed with different amounts of PFSUVs-DOX in a 96-well plate (Greiner bio-One™, Germany), incubated at 37° C. for 30 min and centrifuged at 5000 g for 10 min at 4° C. The supernatant was collected and measured for the absorbance at 540 nm using a microplate reader (Hidex Sense™, Hidex, Finland). PBS and Triton-X 100 (10%) were used as the negative control and positive control, respectively. Relative hemolysis (RH) of PFSUVs was calculated using the equation below:

$$RH \% = \frac{Rs - Rn}{Rp - Rn}$$

Where Rs, Rn and Rp are the absorbance readings of PFSUVs-DOX, negative control and positive control, respectively.

Cryo-Transmission Electron Microscopy (Cryo-TEM) Imaging

The morphology of the empty PFSUVs was imaged by a FEI Tecnai G20 Lab6 200 kV TEM™ (FEI™, Hillsboro, Oreg.) following the method described previously (Belliveau, Huft et al. 2012). The instrument was operated at 200 kV in bright-field mode. Digital images were recorded under low dose conditions with a high-resolution FEI Eagle 4 k CCD™ camera (FEI™, Hillsboro, Oreg.) and analysis software FEI TIA™. A nominal under focus of 2-4 μm was used to enhance image contrast. Sample preparation was performed using the FEI Mark IV Vitrobot™. Approximately 2-4 μL of PFSUVs at ˜ 20 mg/mL total lipid was applied to a copper grid and plunge-frozen in liquid ethane to generate vitreous ice. The frozen samples were then stored in liquid nitrogen until imaged. All samples were frozen and imaged at the UBC Bioimaging Facility (Vancouver, BC).

Preparation of PLD and its Characterization

The thin-film hydration method was utilized to prepare PLD as described before with some modifications (Belliveau, Huft et al. 2012). Briefly, 32 mg of lipid (DSPC/Chol/DSPE-PEG2000=38/25/4, molar ratio) was dissolved in chloroform. The organic solvent was then removed by rotary evaporation (BUCHI™, Flawil Switzerland) at 60° C. The thin film was hydrated with 250 mM ammonium sulfate at 60° C. for 45 min and then sonicated for 10 min with a water-bath ultrasound. The lipid suspension was extruded through 100 nm and 50 nm Nuclepore Track-Etch Membrane™ (Sigma™, Laramine, Wyo.) for 10 times successively using a mini extruder (Avanti Polar Lipids, Inc.™ Alabaster, Ala.). Liposomes were dialyzed (1:1000, volume ratio) against HEPES-buffered saline (HBS, pH 7.4) overnight afterwards. The final lipid concentration of liposomes was determined by a cholesterol assay kit.

One mg DOX was mixed with 8 mg (total lipid) empty liposomes at a total volume of 1 ml adjusted by HBS. The loading mixture was incubated at 60° C. for 45 min and then quenched on ice for another 2 min. Free DOX was then removed by dialysis (1:1000, volume ratio) against HBS for 8 h. PLD was subsequently filtered through 0.22 μm membrane for sterilization. The final concentration of DOX in PLD was measured by the fluorescence (excitation: 485 nm; emission: 590 nm) and compared with a standard curve. PLD was characterized for its size, polydispersity index (PDI), and zeta potential by a Zetasizer™.

In Vitro Drug Retention

PLD and PFSUVs-DOX were adjusted their DOX concentration to 50 μg/ml by sterile PBS, mixed with 1:1 sterile FBS, and then incubated at 37° C. At selected time points, 10 μl of the sample was collected and diluted with PBS for 30-fold. The diluted sample was transferred to a 96-well plate (225 μl sample+25 μl PBS) for fluorescence detection using a microplate reader (Ex 485 nm/Em 595 nm). The percentage of drug retention at each time point was calculated as [1−(Fₜ−F₀)/(Fₜ−F₁₀₀)]×100%, in which Fₜ is the fluorescence at each selected time point, F₀ is the fluorescence at time 0 and F₁₀₀ is the fluorescence of the sample prepared by mixing 225 μl diluted sample and 25 μl Triton-X 100™ (10%), followed by incubation at room temperature for 15 min in dark.

Cell Culture

EMT6 (murine breast tumor) and the resistant variant, EMT6/AR1 cells overexpressing P-glycoprotein were purchased from the National Cancer Institute (Bethesda, Md.). EMT6 cells were cultured in DMEM medium with 10% FBS, penicillin (100 U/ml) and streptomycin (100 μg/ml) at 37° C. with 5% CO₂.

In Vitro Cytotoxicity

EMT6 cells were seeded on a 96-well plate (1000 cells/well). Wells with medium only were used as blank. After 24 h of incubation, cells were treated with different concentrations of free DOX, PFSUVs-DOX and PLD. Two days later, 5 μl of MTT solution (5 mg/ml) was added to each well, followed by 4-h incubation. The medium was removed and 100 μl DMSO was added into each well, followed by incubation at room temperature for 15 min. Absorbance at 540 nm in each well was then measured by a plate reader. The cell viability was calculated as (Abₛ−Ab_{blank})/(Ab₁₀₀−Ab_{blank})×100%, where Abₛ is the Absorbance_{540 nm} for the experimental group, Ab_{blank} is the Absorbance_{540 nm} of sample without cells and Ab₁₀₀ is the Absorbance_{540 nm} of sample without treatment.

Cellular Uptake

Cellular uptake of DOX was imaged by confocal laser scanning microscopy (CLSM). EMT6 cells were seeded on a cover slip placed in a 24-well plate (1×10⁵ cell/well) for 24 h prior to the study. Cells were treated with DOX, PLD or PFSUVs-DOX at a concentration 5 μg DOX/ml in the presence or absence of 10% FBS for 4 h. The medium was removed and the cells were washed with PBS twice before fixation with 10% of formaldehyde at room temperature for 20 min. The cover slip was then washed for another 2 times with PBS and mount on a glass slide with fluorescence shield containing DAPI. The cells were imaged under a Zeiss™ confocal microscope (LSM 700™) and the image was analyzed using the CellProfiler™ (Version 3.0) software.

Cellular Uptake in the Presence of Apolipoprotein

EMT6 cells were seeded in 12 wells on cover slip over night to be confluent. 5 μg/ml Doxorubicin (DOX), PFSUVs-DOX and PLD (reverted into dox concentration) were incubated with EMT6 cells for 4 hours under different conditions including medium, 10% FBS, low concentration of apolipoprotein (5 μg/ml), middle concentration of apolipoprotein (20 μg/ml) and high concentration of apolipoprotein (100 μg/ml). After treatment, the cover slips were fixed with 10% and were then mounted on slide with a DAPI contained fluorescence shield. The slides were imaged under a confocal microscopy. Doxorubicin signal in those images were then quantified by Cellprofiler™.

Mice

Female Balb/c mice (6-8 weeks old) purchased from the Jackson Laboratory™ (Bar Harbor, Me.). All animal studies were conducted with approved protocols in compliance with the guidelines developed by the Canadian Council on Animal Care.

Subcutaneous EMT6 Tumor Model

Approximately 1×10⁵ EMT6 cells were subcutaneously injected to right flank of BALB/C mice. Mice were subjected for in vivo studies when the tumor reached a volume of ˜200 mm³.

Pharmacokinetics

PLD and PFSUV-DOX (5 mg DOX/kg) were administered to tumor-bearing mice via tail vein injection. Mice were euthanized at various time points. About 100 μl blood was collected from mice by cardiac puncture. Plasma was immediately isolated by centrifugation of the blood at 4° C. for 15 min at 2,500 rpm. The plasma concentration of DOX was measured by a previously reported method (Tagami, Ernsting et al. 2011). Briefly, 10 μl of plasma was diluted with 990 μl acidified isopropanol (IPA) and the mixture was incubated at 4° C. in the dark for overnight. The sample was then centrifuged for 10 min at 12,000×g and the supernatant was loaded onto a 96-well plate for fluorescence determination (Eₓ 485 nm/Eₘ 595 nm). The plasma concentration was then obtained by comparing the fluorescence with a calibration curve generated by spiking known amounts of DOX into mouse plasma.

Biodistribution

After the euthanasia of the mice, different tissues including heart, liver, spleen, kidney, lung, tumor and brain, were excised. The experimental procedures were adapted from the previously published literature (Tagami, Ernsting et al. 2011). The tissue was washed with PBS, weighed after removing excess fluid and put into a 1.5-ml microtube. Normally, 0.1-0.3 g tissue was collected. The nuclear lysis buffer (10 mM HEPES, 1 mM MgSO₄, 1 mM CaCl₂, pH 7.4) with a volume three times to the tissue weight was added into the microtube, and tissue homogenization was performed for 2×30 s at 6,600 rpm with a tissue homogenizer (Precellys 24™, Bertin Technologies™, Cartland, Calif.). An aliquot of the homogenate (100 μl) was transferred into a 1.5 ml microtube, and 50 μl of 10% (v/v) Triton X-100™, 100 μl of water, and 750 μl of acidified IPA were added and the mixture was stored for overnight at −20° C. The mixture was then thawed, equilibrated at room temperature for 1 h, centrifuged for 10 min at 12,000×g, and the supernatant was loaded onto a 96-well plate (Eₓ 485 nm/Eₘ 590 nm) for DOX determination. The data was compared with standard curves made from spiking known amounts of DOX into different tissue homogenates from the untreated mice to get the absolute quantification of DOX in different tissues.

Tissue Section

Liver in the PFSUVs-DOX treated mice was harvested 2 h post injection, fixed in 10% formaldehyde, sectioned using a Vibratome™ (Precisionary Instruments™, Boston, Mass.). Tissue sections with a thickness of 40 μm were collected in PBS and then stained with fluorescien-phalloidin (40 U/ml) for 15 min at room temperature. The sections were imaged under confocal microscopy.

PFSUVs Loaded with Chloroquine, Imiquimod, R848, Curcumin, and Sodium Diatrizoate

PFSUVs (2.0 mg total lipids) were incubated with 100 μg drug in the appropriate buffer (final volume 1 mL). Chloroquine diphosphate (Sigma-Aldrich™) was loaded in HBS (pH 7.4), whereas imiquimod and R848 (both Cayman Chemical™, Ann Arbor, Mich., USA) were loaded in 100 mmol/l sodium acetate buffer (pH 5). The mixture was incubated for 1 h at 37° C. and then quenched on ice for 2 min. The drug-loaded particles were subjected to purification by TFF as described above in the diafiltration mode using ten diafiltration volumes of buffer. The encapsulated contents of chloroquine, imiquimod, and R848 were determined using ultra performance liquid chromatography (UPLC). PFSUVs (20 μl) were lysed by adding 40 μl methanol (VWR™, Mississauga, ON, Canada) and sonication (5 min). Samples were analyzed on an ACQUITY UPLC H-Class System (Waters, Milford, Mass.) coupled online to a photodiode array detector. Separation relied on a BEH-C18 column (inner diameter: 2.1 mm; length: 50 mm; particle size: 1.7 μm, Waters™; column temperature: 60° C.) at a flow rate of 0.3 mL min-1 using a linear aqueous methanol gradient in the presence of trifluoroacetic acid (TFA, ≈98%, Alfa Aesar™, Tewksbury, Mass.). Eluent A and B consisted of 0.1% v/v aqueous TFA and methanol containing 0.1% v/v TFA, respectively, and were mixed in the following gradient. 1 min: A/B (95/5); 6 min: A/B (0/100); 3 min: A/B (0/100); 1 min: A/B (95/5); 2 min: 1 min: A/B (95/5). Drugs and cholesterol were detected via absorbance at 342 nm (chloroquine), 320 nm (imiquimod and R848), and 205 nm (cholesterol), respectively, and quantified using calibration curves to calculate drug loading values. The encapsulation efficiency was calculated as a ratio of drug loading values before and after purification of the freshly loaded particles. Curcumin (Alfa Aesar™) was encapsulated into PFSUVs at a D/L of 1/40 via a passive loading approach during their preparation. Chol, TWEEN 80, and curcumin at a molar ratio of 72.5:25:2.5 were dissolved in ethanol at a final concentration of 10 mg/ml. This solution was mixed with PBS in the microfluidic system at a flow ratio of 1/3 between ethanol and the aqueous phase. The setting of the microfluidic preparation process and purification was as described above. The encapsulation efficiency of curcumin-loaded particles was determined using UPLC as described above with detection of curcumin at an absorbance of 430 nm. Sodium diatrizoate was encapsulated into PFSUVs via passive loading approach during their preparation. Chol, TWEEN 80 was dissolved at a molar ratio of 5:1 were dissolved in ethanol at a final concentration of 10 mg/ml. This solution was mixed with aqueous sodium diatrizoate (500 g/L, Sigma Aldrich™) in the microfluidic system at a flow ratio of 1/3 between ethanol and the aqueous phase. The setting of the microfluidic preparation process and purification was as described above. The encapsulation efficiency of diatrizoate-loaded particles was determined using UPLC as described above with detection of diatrizoate at an absorbance of 238 nm.

Statistics Analysis

All data are expressed as mean f SD. Statistical analysis was conducted with the two-tailed unpaired t test for two group comparison or one-way ANOVA, followed by the Turkey multiple comparison test by using GraphPad Prism™ (for three or more groups). A difference with p<0.05 was considered to be statistically significant.

The inventors herein further describe the present invention byway of the following non-limiting examples:

### EXAMPLES

### Example 1: The Effect of the Cholesterol/Tween 80 Ratio on the Size and Stability of PFSUVs

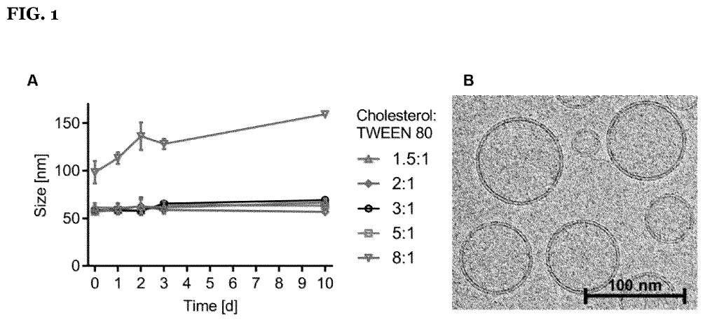

PFSUVs with different cholesterol/Tween 80™ ratios were formulated using microfluidics. As shown in FIG. 1, the cholesterol/Tween 80™ ratio had minimal effect on the size as most formulations exhibited a mean diameter between 60 and 70 nm. However, when the cholesterol ratio increased to approximately 90% (with the 8:1 ratio of Cholesterol:Tween 80™), the particle size increased to 115.0±5.6 nm. The stability of PFSUVs at 4° C. was monitored by measuring the size over time. No visible precipitates were spotted in the PFSUV formulations after 10 days of storage (data not shown). All the formulations were stable for 10 days except for the one composed of cholesterol/Tween 80™ (8:1) where and the size increased from 115.0±5.6 nm to 176.6±21.2 nm (as shown in FIG. 1). The increase in size that was observed with cholesterol/Tween 80™ (8:1 ratio), could be due to precipitation of cholesterol in the bilayer when the content reached 88 mol % of the bilayer composition. These data are in contrast previous studies where niosomes were only stable at a low cholesterol content (50 mol % [1:1] or below) (Kazi, Mandal et al. 2010; and Taymouri and Varshosaz 2016, Bartelds, Nematollahi et al. 2018). This could suggest that the preparation method (thin-film hydration as compared to microfluidics) may affect the formation of the phospholipid-free bilayer and disperse the cholesterol component more rapidly and efficiently, leading to improved stability. During the microfluidic process, rapid mixing of the ethanol/lipid solution with the aqueous phase results in a rapid increase in the polarity of the medium, which causes the solution to quickly achieve a state of high super-saturation of lipid monomers throughout the entire mixing volume (Uchegbu, Double et al. 1996), leading to rapid and homogeneous nucleation of lipid bilayer. These nucleation events are very rapid (<1 ms) compared to the time-scale for particle formation (Uchegbu, Double et al. 1996).

### Example 2: Drug Loading Optimization and Kinetics for PFSUVs

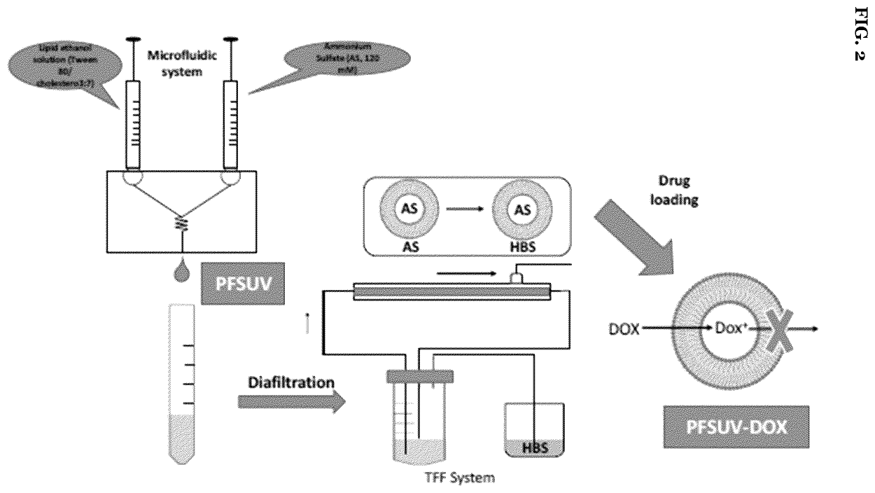

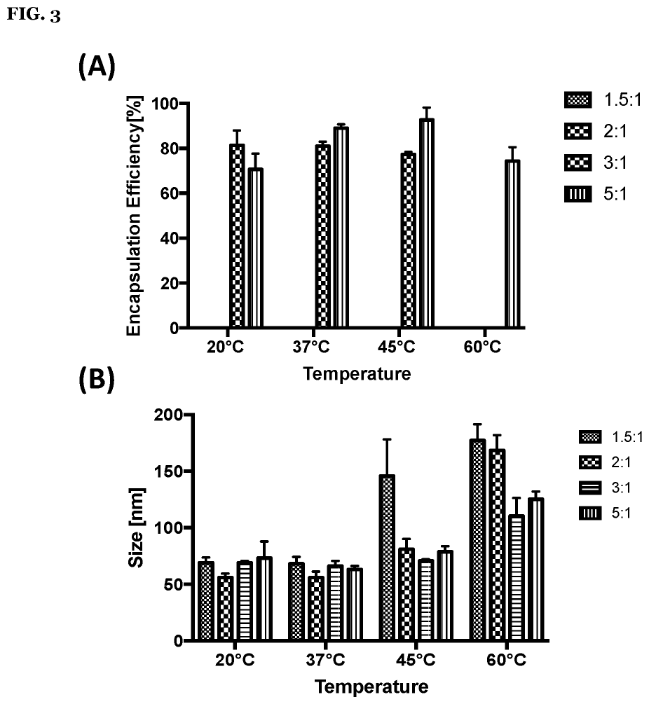

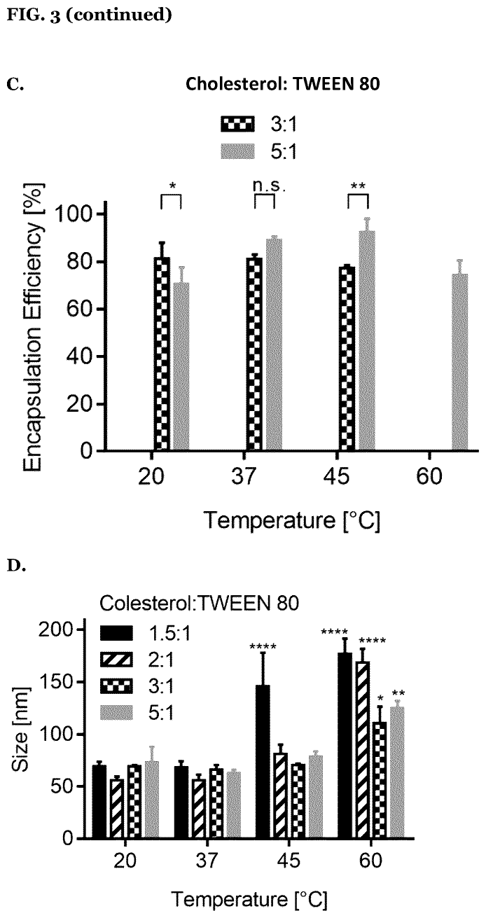

We then investigated whether doxorubicin (DOX) could be actively loaded into different PFSUV formulations (method as shown in FIG. 2) and whether the incubation temperature affected the encapsulation efficiency and the particle stability. As shown in FIG. 3A, the EE % was impacted by the lipid formulation and loading temperature. Only formulations containing a lower amount of Tween 80™ (3:1 and 5:1) could load DOX via an active mechanism. For the 3:1 formulation, the EE % at 20-45° C. was comparable (˜80%), while there was no drug loading at 60° C. On the other hand, the drug encapsulation efficiency for the 5:1 formulation displayed an increasing trend (from 70% to 90%) with increasing temperature (from 25° C. to 45° C.), except that at 60° C. the EE % declined back to 70%. As shown in FIG. 3B, the incubation temperature could also affect drug loading; when loading at 37° C. or below, there was no change in size of the final particles. However, when the incubation temperature increased to 45° C., the high Tween80™ formulation (1.5:1) displayed a significant increase in size to ˜150 nm. When the loading temperature further increased to 60° C., the particle size increased in all the formulations (100-170 nm). As shown in FIGS. 3C and 3D, DOX was also actively loaded in PFSUVs using a citric acid gradient with comparable results to the ammonium sulfate gradient. This indicates significant potential of PFSUVs for maintaining various transmembrane gradients for loading of different agents.

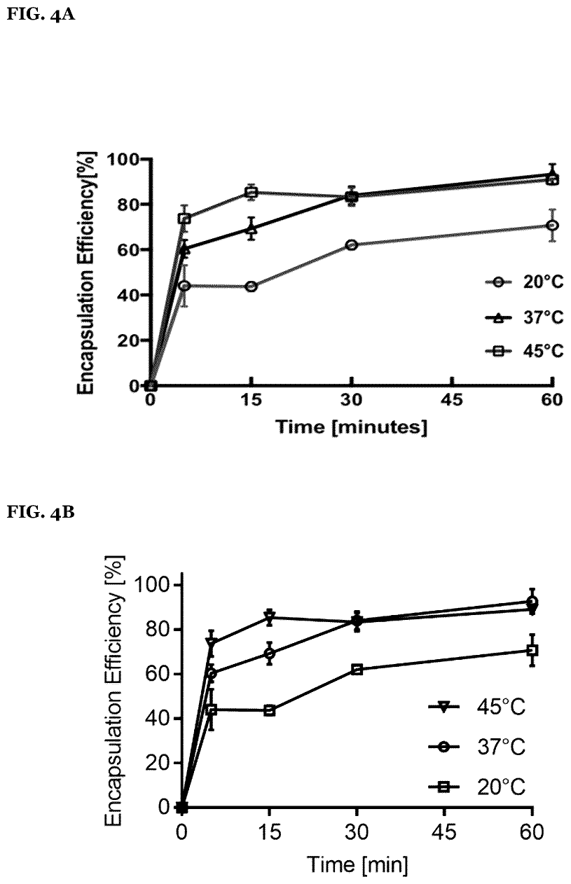

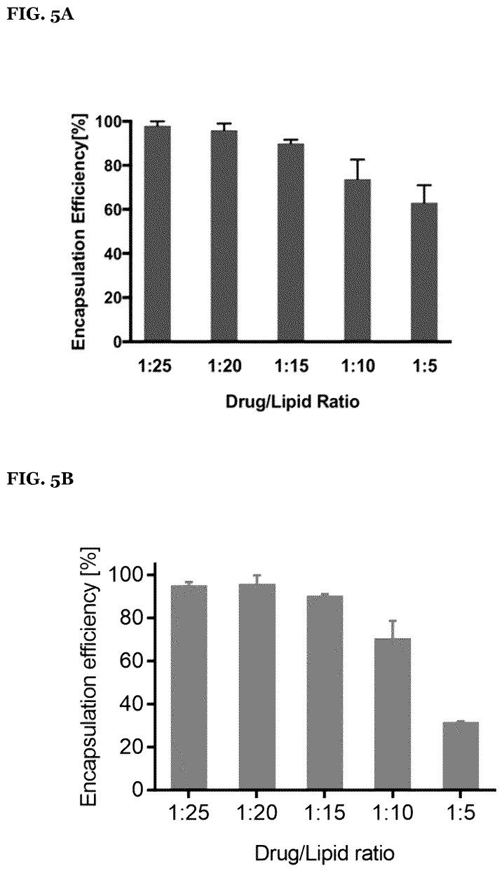

DOX EE % at different time points under different incubation temperature was measured. As shown in FIG. 4, the drug loading kinetics was dependent on the loading temperature and incubation time. As the incubation temperature increased, the drug EE % increased and reached the maximum faster. For example, at 20° C., the EE % slowly reached the maximum at ˜60% after 30-60 min of incubation, while the loading reached the plateau of ˜90% in 15 min when incubated at 45° C. At 37° C., it took 30-60 min to achieve ˜90% of drug loading. To investigate the loading capacity of the PFSUVs, drug EE % at different drug-to-lipid ratio (D/L) was compared. As shown in FIG. 5, the drug EE % gradually decreased as D/L increased, and the highest D/L was 1/20 for complete drug loading (>95%).

In summary, doxobubicin (DOX) (a weak base drug) can be actively loaded into preformed liposomes using an ammonium sulfate gradient (inner core: 250 mM ammonium sulfate, pH5; outer phase: HEPES buffered saline pH 7.4). It was found that the use of a high temperature incubation significantly increases the lipid membrane permeability resulting in the permeation of non-ionized form DOX into the liposomal core. Under this acidic environment in liposome, DOX is protonated and no longer membrane permeable. The protonated DOX can form complexes with the sulfate ion inside the core, generating insoluble precipitates inside the liposomes. This irreversible process drives effective loading and DOX precipitation inside the liposomal core leading to reduced drug leakage. The Active DOX loading into niosomes has never been previously reported, as it was previously thought that a phospholipid-free bilayer (cholesterol:surfactant ratio of 5:5 formulation) was not sufficiently stable to maintain the loading gradient. As we obtained PFSUVs with a range of cholesterol/Tween ratio, we tested their ability to maintain a loading gradient of 120 mM ammonium sulfate. Indeed, when the cholesterol content was below 75 mol % [3:1], no DOX loading was measured under all the tested conditions, while >80% DOX could be actively loaded into PFSUVs containing 75% and 83% [3:1 and 5:1] Tween80™ when incubated at 20-45° C. Interestingly, when incubated at 60° C., no DOX loading into the 3:1 formulation was measured, while 75% loading efficiency was obtained within the 5:1 formulation, suggesting heating could disrupt the membrane integrity for a formulation containing an increased amount of surfactant. Therefore, our data indicate the optimal PFSUVs formulation was 5:1 cholesterol:Tween80™, which exhibited a small (60-80 nm) and stable particle size. The formulation is capable of maintaining a loading gradient for active encapsulation of DOX under a wide range of conditions. Finally, the loading kinetics of the PFSUVs followed a similar pattern as the regular liposomes, for which as the incubation temperature increased the rate and amount of drug loading increased. To determine whether the loading gradient could impact the encapsulation efficiency, we compared PFSUVs loaded with DOX with a loading gradient of either ammonium sulfate or citric acid. As shown in TABLE 1, the two loading gradients showed similar EE % (>90%) at drug/lipid ratios of 1:25 to 1:15, whereas the EE % decreased when the drug/lipid ratio increased (1:10 and 1:5).

TABLE 1
The effect of the Drug/Lipid Ratio on Encapsulation Efficiency
for PFSUV-Dox loaded with either a citric acid gradient
(300 mM) or an ammonium sulfate gradient (120 nM)

| Loading gradient | Drug:Lipid Ratio | Encapsulation Efficiency % |
|---|---|---|
| Citric Acid | 1:25 | 96.6 |
| Ammonium Sulfate | 1:25 | 98 |
| Citric Acid | 1:20 | 95.3 |
| Ammonium Sulfate | 1:20 | 95 |
| Citric Acid | 1:15 | 91.7 |
| Ammonium Sulfate | 1:15 | 90.7 |
| Citric Acid | 1:10 | 68.3 |
| Ammonium Sulfate | 1:10 | 85 |
| Citric Acid | 1:5 | 31 |
| Ammonium Sulfate | 1:5 | 63 |

The cryo-TEM images of PFSUVs showed a phospholipid-free bilayer with DOX crystalline loaded inside the aqueous core. Thus, the cholesterol:Tween80™ (5:1) formed a bilayer structure that could maintain a gradient for active loading of DOX. Interestingly, the PFSUVs remained their spherical shape after DOX loading, while PLD displayed an oval morphology due to the big size DOX crystalline inside the liposomes. This can be explained by that the D/L in the PFSUVs was only ˜1/3 of that in the PLD, and the small size of DOX crystalline did not alter the SUV shape.

### Example 3: Characterization of the PFSUVs

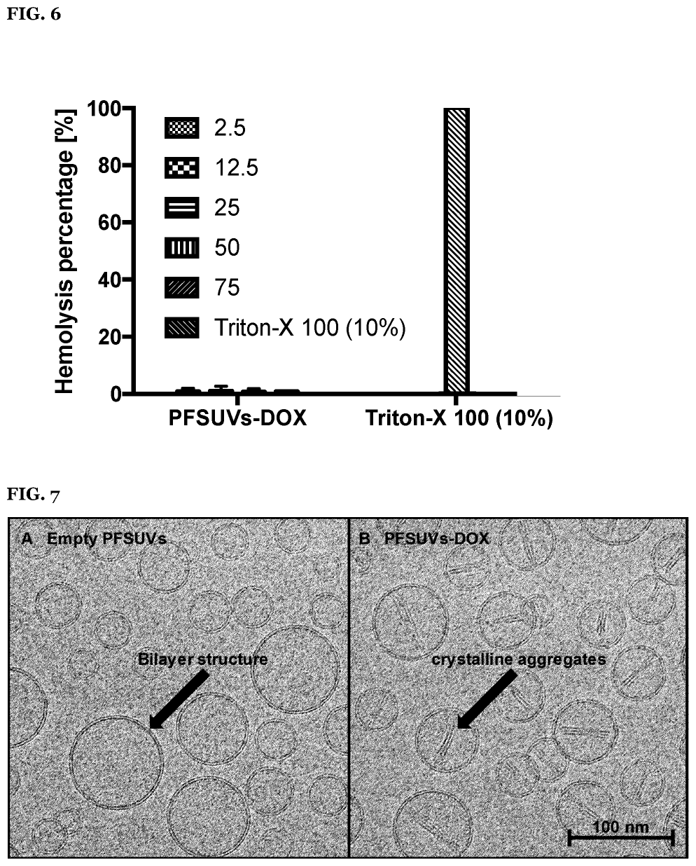

Hemolytic toxicity of the PFSUVs-DOX was measured by incubating the formulation at different concentrations with SRBC. As shown in FIG. 6, PFSUVs exhibited little hemolytic toxicity even when loaded with increasing concentrations of DOX. As shown in FIG. 7, the empty PFSUVs displayed a bilayer structure with a spherical morphology (panel A) and DOX crystalline was found in the aqueous core of the SUVs (panel B). The formation of DOX crystalline inside the PFSUVs did not alter the particle morphology compared to the empty vehicle.

Both PLD and PFSUVs-DOX were characterized by the size, PDI, zeta potential (ZP) and EE %. Their formulation parameters are compared in TABLE 1. PFSUVs-DOX prepared by microfluidics were significantly smaller than the PLD fabricated by membrane extrusion. PFSUVs-DOX exhibited neutral surface charge with a ZP close to 0 mV, while the PLD displayed negatively charged surface (−25 mV). The PLD provided an increased D/L compared to PFSUVs-DOX, indicating a higher drug content per particle.

TABLE 2
comparison between PFSUVs-DOX and PLD.

| | | | D/L | Size | | ZP | EE |
|---|---|---|---|---|---|---|---|
| Formulation | Composition | Method | ratio | (nm) | PDI | (mV) | % |
| PFSUV-DOX | Tween 80/ | microfluidic | 1:20 | 74.67 ± 4.51 | 0.132 ± 0.027 | −5.49 ± 3.68 | 96 ± 3.2 |
| | Cholesterol | | | | | | |
| | (1/5) | | | | | | |
| PLD | DSPC/DSPE- | Thin-film | 1:8 | 111.3 ± 9.67 | 0.036 ± 0.023 | −24.7 ± 1.9 | 95 ± 1.1 |
| | mPEG2000/ | extrusion | | | | | |
| | Cholesterol | | | | | | |
| | (38/4/25) | | | | | | |

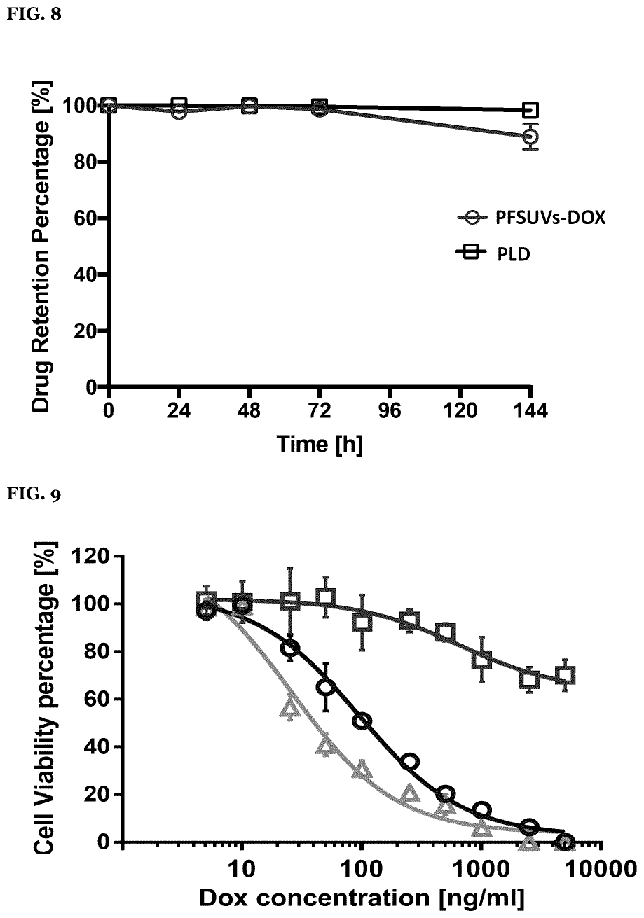

A comparison of drug retention profiles can conducted as shown in FIG. 8. Briefly, during the first 3 days of incubation with 50% FBS, no DOX release was detected from either PFSUVs-DOX or PLD. Six days later, approximately 10% of DOX was released from PFSUVs-DOX, while no drug leakage was measured with PLD.

### Example 4: Intracellular Uptake and Antitumor Efficacy of Drug-Loaded PFSUVs

Intracellular delivery of DOX by different formulations was analyzed by CLMS imaging, and the images were quantified by CellProfiler™. Uptake by the EMT6 cells in the absence of serum, showed that DOX uptake in the PFSUVs-DOX and PLD groups was minimal, while free DOX displayed highly efficient co-localization with the nucleus (data not shown). In the presence of serum, there was a significant increase in DOX uptake in the PFSUVs-DOX group compared to the serum free conditions. The presence of serum did not significantly change the DOX uptake in free DOX and PLD groups. The quantitative data showed that free DOX displayed ˜15-fold increased cellular uptake relative to PFSUVs-DOX and PLD in the absence of serum, while the intracellular delivery of PFSUVs-DOX was increased by 2-fold in the presence of serum (data summarized below in TABLE 3).

TABLE 3
Mean fluorescence intensity in each cell after 4 hr
treatment with either DOX, PLD or PFSUV-DOX. The
results were quantified using cell profiler software
on the result of confocal imaging (N > 30).
Mean Fluorescence Intensity

| | Without Serum | 10% Serum |
|---|---|---|
| PFSUVs-DOX | 0.015 ± 0.002 | 0.029 ± 0.004 |
| PLD | 0.016 ± 0.003 | 0.015 ± 0.001 |
| DOX | 0.258 ± 0.014 | 0.247 ± 0.021 |

In vitro cytotoxicity of free DOX, PFSUVs-DOX and PLD against EMT6 murine breast cancer cells was evaluated by MTT assay, and the IC₅₀ values were obtained by curve fitting using GraphPad™. As shown in FIG. 9, the curves of free DOX and PFSUVs-DOX were largely overlapping, suggesting comparable potency, while the PLD was significantly less potent in inhibiting EMT6 cells. The IC₅₀ values for free DOX, PFSUVs-DOX and PLD were 25, 94 and 1658 ng/ml, respectively. PLD was 20-fold less effective compared to PFSUVs-DOX for the in vitro potency and DOX indicated 4-fold more effective than PFSUVs-dox for the in vitro potency.

In summary, the PFSUV-DOX cellular uptake increased by ˜2 fold in the presence of serum, and after a 4 hr incubation, DOX could be detected in the nucleus of EMT6 cells treated with PFSUV-DOX.

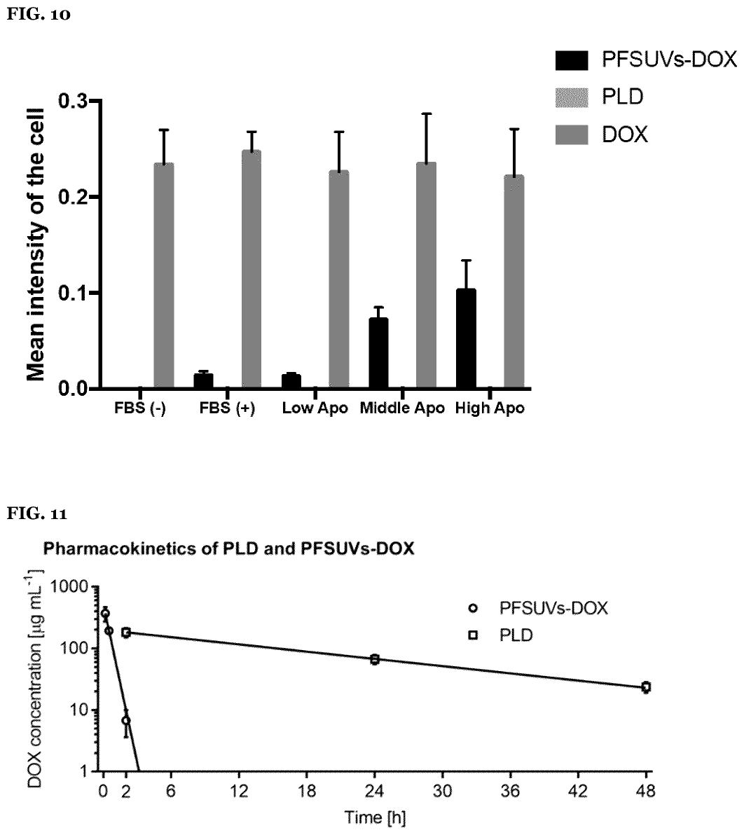

Next, we investigated whether the presence of apolipoprotein in the media would have an impact of cellular uptake of the particles. As shown in FIG. 10 EMT6 cells incubated in the presence of increasing concentrations of apolipoproteins had a higher rate of internalization of the PFSUV-DOX particles, whereas the presence of apolipoprotein had no influence on the internalization rate for PLD or free DOX. This could suggest cellular uptake via the LDL endocytosis pathway.

### Example 5: Pharmacokinetics and Biodistribution of PFSUVs

For the in vivo studies, an ultrafiltration method was first used to concentration the PFSUV-DOX preparation, but it led to disruption of the particles, possibly due to the collapse of the vesicles onto the membrane by high centrifugation force. Therefore the TFF system was utilized allowing simultaneous ethanol removal, buffer exchange and particle concentration under gentle and controlled conditions. In the TFF system, the particle flow was in parallel with the diafiltration membrane, thus reducing the collapse of the particles onto the membrane.

Plasma concentration of DOX was measured at different time points after an i.v. injection of PFSUVs-DOX and PLD and was plotted as shown in FIG. 11. PLD displayed a prolonged plasma circulation profile and the plasma concentration declined slowly from 2 h (183.5 μg/ml) to 48 h (37.2 μg/ml). DOX in the PFSUVs-DOX treated mice could only be detected 2 h post injection (4.7 μg/ml), indicating PFSUVs-DOX were rapidly removed from the plasma.

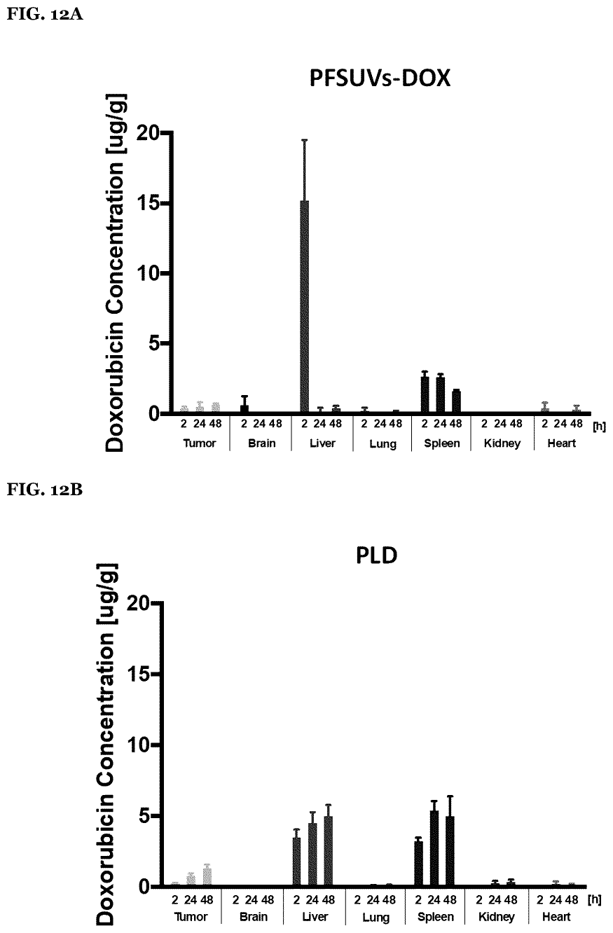

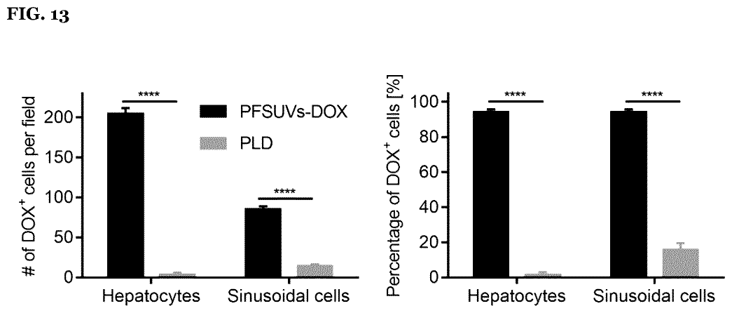

DOX concentration in different tissues at different time points after treatment with PFSUVs-DOX or PLD was measured and reported in FIG. 12. The PLD formulation selectively accumulated in the tumor, liver and spleen (>1.5 μg/g tissue), and displayed minimal uptake by other tissues, including the brain, lung, kidney and heart (<0.3 μg/g tissue). The data also showed that there was a gradual increase of PLD uptake in the tumor, liver and spleen from 2-48 h. In 48 h, PLD uptake in these tissues reached the maximum with 1.3 μg/g, 5.7 μg/g and 5.4 μg/g measured in the tumor, liver and spleen, respectively. On the other hand, PFSUVs-DOX showed early uptake (2 h) in tissues, including the tumor, brain, liver and spleen, but the concentration rapidly declined to background in the brain and liver. The tumor uptake of PFSUVs-DOX stayed consistently from 2-48 h at 0.34 μg/g, and the spleen uptake only dropped from 5 μg/g to 2.5 μg/g from 2 h to 48 h. The most significant uptake of PFSUVs-DOX occurred in 2 h in the liver, showing ˜15 μg/g, but rapidly decreased to almost undetectable in one day. To examine what cells in the liver contributing to the uptake of PFSUVs-DOX, the liver was collected 2 h after injection, sectioned and imaged. As shown in FIG. 13, significant PFSUV-DOX fluorescence was detected in both hepatocytes and sinusoidal cells, whereas PLD was primarily detected in sinusoidal cells at a reduced level.

The pharmacokinetic and biodistribution profiles of PFSUVs-DOX were distinctive from the PLD. PFSUVs-DOX was short-lived in the plasma and only a minimal DOX concentration could be detected in the plasma 2 h post injection. In the biodistribution results, it was shown that PFSUVs-DOX were largely taken up the by the liver and removed from the blood circulation. The uptake by the other examined tissues was only minimal, suggesting this formulation targeted the liver in high efficiency. Additionally, the drug was largely delivered to the hepatocyte rather than the Kupffer cells. Again, this could be explained by several factors. First, PFSUVs-DOX were 60-80 nm in size, which could easily pass the liver fenestrae (mean size ˜100 nm) to reach the hepatocytes [Braet F, Wisse E. 2002]. Second, the blood flow to the liver is high and this would bring a large dose of PFSUVs-DOX to the liver. Third, hepatocyte is known to overexpress LDL receptor and the Tween80/ApoE/LDL-receptor mechanism described above would help the internalization of PFSUVs-DOX. The results indicated that this liver-targeted formulation may be used to deliver other drugs for treating liver diseases. It is also interesting to see that the liver uptake of DOX rapidly declined to the background 24 h post injection, which could be due to that DOX is a substrate for P-glycoprotein that is highly expressed in the hepatocyte and that DOX would be rapidly removed from the hepatocytes by the efflux pump. Similarly, DOX delivered by PFSUVs was detected in the brain 2 h post injection but rapidly cleared. The data could be justified by the same reasons mentioned above that the brain endothelial cells (so called blood-brain barrier) overexpress LDL receptor and P-glycoprotein. Our data also showed that because there was no prolonged circulation of PFSUVs-DOX, the tumor accumulation did not increase over time.

In summary, novel PFSUV formulations (60-80 nm) were developed with high cholesterol content using a microfluidic method for manufacturing. This is the first time that a surfactant-based formulation with cholesterol content over 80% has been reported. Even with this high cholesterol concentration, a bilayer structure was still observed by the cryo-TEM, allowing active loading procedure for DOX and a stable retention in the PFSUVs via an ammonium gradient or citric acid gradient. PFSUVs-DOX displayed significantly different profiles of pharmacokinetics and biodistribution compared to PLD, and were demonstrated to be hepatocyte-targeting in mice.

Using the microfluidic-based method, the fabricated PFSUVs overcome a number of fabrication challenges that are encountered with traditional niosomes, including difficulties in homogenous hydration and efficient membrane extrusion for size control. Previously, it was thought that the phospholipid-free bilayer would be very leaky and would not maintain a loading gradient. However, the high cholesterol concentration can play a role to retain the gradient for active loading. Therefore, it is of interest to explore whether this formulation would be compatible with different loading gradients for active encapsulation of drugs for various applications, including for drugs or imaging agents useful for treatment and/or diagnosis and monitoring of diseases affecting the liver. In addition, the data also indicated that PFSUV-DOX exhibited increased brain uptake, and it was reported that the LDL receptor mediated transcytosis has been utilized for drug delivery to the brain (Wang, Meng et al. 2015). In addition to the studies here with Tween80™, other nonionic surfactants may be used to replace Tween80™ to generate formulations that may display unique biodistribution profiles for medical applications.

### Example 6: Chloroquine, Imiquimod, R848, Curcumin and Sodium Diatrizoate Loading of PFSUVs

TABLE 4
Physical properties of PFSUVs loaded with different drugs.

| | | | | Drug | | |
|---|---|---|---|---|---|---|
| PFSUVs- | Loading | D/L | EE | loading value | Diameter | |
| Formulation | method | ratio | [%] | [w %] | [nm] | PDI |
| chloroquine | Active | 1:20 | 95.4 ± 2.2 | 4.8 ± 0.1 | 57.0 ± 0.4 | 0.13 ± 0.01 |
| imiquimod | Active | 1:20 | 98.2 ± 4.7 | 4.9 ± 0.3 | 57.5 ± 3.4 | 0.13 ± 0.03 |
| R848 | Active | 1:20 | 93.3 ± 8.1 | 4.7 ± 0.5 | 50.1 ± 0.8 | 0.09 ± 0.02 |
| curcumin | Passive | 1:40 | 88.9 ± 8.6 | 2.2 ± 0.2 | 56.4 ± 0.6 | 0.10 ± 0.04 |
| sodium | Passive | 1:4 | 0.23 ± 0.05 | 25.8 ± 4.0 | 67.8 ± 0.6 | 0.19 ± 0.05 |
| diatrizoate | | | | | | |

Data = mean ± SD (n = 3).

Liver diseases are a global health problem accounting for Z; 2 million deaths per year worldwide, including viral hepatitis, non-viral hepatitis, cholestatic liver disease, non-alcoholic steatohepatitis (NASH), non-alcoholic fatty liver disease (NAFLD), primary biliary cholangitis, liver fibrosis, biliary atresia, parasitic infections of the liver and genetic liver diseases such as hemochromatosis, Wilson's disease, alpha-i antitrypsin deficiency, hyperoxaluria, oxalosis liver cirrhosis, hepatitis B, and hepatocellular carcinoma [Asrani et al. 2019; Mokdad et al. 2014]. Both sinusoidal cells and hepatocytes are crucially involved in these diseases.

Current nanoparticle delivery technologies mainly target Kupffer cells that represent 10%-15% of the liver cells [Zhou et al. 2016; Li et al. 2016]. Drug delivery systems that also target other types of liver cells, including the hepatocytes, the dominant liver cells (≈60%), will be highly desirable for improving therapy of liver diseases. As PFSUVs can maintain transmembrane gradients for active loading of drugs, making this formulation attractive for targeting a wide range of therapeutic agents to treat various liver disorders. Additionally, PFSUVs may be used for active loading of imaging agents to diagnose or provide a disease stratification for liver disorders. Examples of imaging agents for the liver can include radiolabeled agents for positron emission tomography, single photon emission computed tomography and magnetic resonance imaging. To show that PFSUVs will be potentially useful for the treatment of major liver diseases, we encapsulated several drugs relevant for liver diseases at the same conditions as optimized for DOX (TABLE 4).

Malaria is characterized with an initial liver stage, where parasite sporozoites invade hepatocytes and undergo asexual replication before progressing to the blood [Raphemot et al. 2016; Li et al. 2016]. Quinone drugs are used to treat malaria and efficient liver targeting to stop malaria progression at the liver stage remains a challenge [Tibenderana et al. 2011]. We encapsulated the weakly basic quinine drug chloroquine using the AS gradient into PFSUVs achieving an EE of 95.4%. Further development of this delivery system encapsulated with chloroquine and other quinine-based drugs such as primaquine could be highly beneficial for liver-stage malaria treatment [Oliver et al. 2008]. Another liver-related infectious disease related to liver impairment is viral hepatitis (hepatitis B and hepatitis C) resulting in liver cirrhosis and hepatocellular carcinoma [Wang et al. 2016]. Immune modulators targeting the Toll-like receptor 7/8 such as imiquimod and resiquimod (R848) have been investigated as an interferon α booster to treat hepatitis C [O'Neill et al. 2010; Tomai et al. 2010]. Both imiquimod and R848 are weakly basic drugs and could be efficiently loaded into PFSUVs (EE: 98.2% and 93.3%, respectively) using the AS gradient. Finally, liver injury induced by a variety of agents such as alcohol, environmental pollutants, dietary components, and drugs, resulting in progression of steatohepatitis, liver fibrosis, or cirrhosis remains a problem in society [Asrani et al. 2019; Mokdad et al. 2014]. Curcumin, a natural product isolated from turmeric, exerts hepatoprotective and therapeutic effects on several liver diseases associated with oxidative stress and inflammation through various cellular and molecular mechanisms [Farzaei et al. 2018]. Nanoformulations of curcumin are an emerging field for improving the bioavailability and organ targeting of this compound [Mehanny et al. 2016]. As a hydrophobic drug, we encapsulated curcumin in the bilayer of PFSUVs via passive loading (EE=88.9% at a D/L of 1/40). Potential in medical applications of these formulations will be demonstrated in future studies.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

Abdelkader, H., A. W. G. Alani and R. G. Alany (2014). “Recent advances in non-ionic surfactant vesicles (niosomes): self-assembly, fabrication, characterization, drug delivery applications and limitations.” Drug Delivery 21(2): 87-100.
Abdelkader, H., U. Farghaly and H. Moharram (2014). “Effects of surfactant type and cholesterol level on niosomes physical properties and in vivo ocular performance using timolol maleate as a model drug.” Journal of Pharmaceutical Investigation 44(5): 329-337.
Abdelkader, H., S. Ismail, A. Kamal and R. G. Alany (2010). “Preparation of niosomes as an ocular delivery system for naltrexone hydrochloride: Physicochemical characterization.” Die Pharmazie—An International Journal of Pharmaceutical Sciences 65(11): 811-817.
Auda, S. H., D. Fathalla, G. Fetih, M. El-Badry and F. Shakeel (2016). “Niosomes as transdermal drug delivery system for celecoxib: in vitro and in vivo studies.” Polymer Bulletin 73(5): 1229-1245.
Bartelds, R., M. H. Nematollahi, T. Pols, M. C. A. Stuart, A. Pardakhty, G. Asadikaram and B. Poolman (2018). “Niosomes, an alternative for liposomal delivery.” PLOS ONE 13(4): e0194179.
Bastiat, G., P. Oliger, G. Karlsson, K. Edwards and M. Lafleur (2007). “Development of Non-Phospholipid Liposomes Containing a High Cholesterol Concentration.” Langmuir 23(14): 7695-7699.
Belliveau, N. M., J. Huft, P. J. Lin, S. Chen, A. K. Leung, T. J. Leaver, A. W. Wild, J. B. Lee, R. J. Taylor, Y. K. Tam, C. L. Hansen and P. R. Cullis (2012). “Microfluidic Synthesis of Highly Potent Limit-size Lipid Nanoparticles for In Vivo Delivery of siRNA.” Mol Ther Nucleic Acids 14(1): 28.
Belliveau, N. M., J. Huft, P. J. Lin, S. Chen, A. K. Leung, T. J. Leaver, A. W. Wild, J. B. Lee, R. J. Taylor, Y. K. Tam, C. L. Hansen and P. R. Cullis (2012). “Microfluidic Synthesis of Highly Potent Limit-size Lipid Nanoparticles for In Vivo Delivery of siRNA.” Mol Ther Nucleic Acids 1: e37.
Braet F, Wisse E. Structural and functional aspects of liver sinusoidal endothelial cell fenestrae: a review. Comp Hepatol. 2002 Aug. 23; 1(1):1. PubMed PMID: 12437787.
Budhiraja, A. and G. Dhingra (2015). “Development and characterization of a novel antiacne niosomal gel of rosmarinic acid.” Drug Deliv 22(6): 723-730.
Fang, J.-Y., C.-T. Hong, W.-T. Chiu and Y.-Y. Wang (2001). “Effect of liposomes and niosomes on skin permeation of enoxacin.” International Journal of Pharmaceutics 219(1): 61-72.
Gaafar, P. M. E. “Preparation, characterization and evaluation of novel elastic nano-sized niosomes (ethoniosomes) for ocular delivery of prednisolone.” Journal of Liposome Research 24(3): 204-215.
Kazi, K. M., A. S. Mandal, N. Biswas, A. Guha, S. Chatterjee, M. Behera and K. Kuotsu (2010). “Niosome: A future of targeted drug delivery systems.” Journal of advanced pharmaceutical technology & research 1(4): 374-380.
Khalil, R. A. and A.-h. A. Zarari (2014). “Theoretical estimation of the critical packing parameter of amphiphilic self-assembled aggregates.” Applied Surface Science 318: 85-89.
Khalil, R. M., G. A. Abdelbary, M. Basha, G. E. A. Awad and H. A. El-Hashemy (2017). “Design and evaluation of proniosomes as a carrier for ocular delivery of lomefloxacin HCl.” Journal of Liposome Research 27(2): 118-129.
Li, Q., Z. Li, W. Zeng, S. Ge, H. Lu, C. Wu, L. Ge, D. Liang and Y. Xu (2014). “Proniosome-derived niosomes for tacrolimus topical ocular delivery: In vitro cornea permeation, ocular irritation, and in vivo anti-allograft rejection.” European Journal of Pharmaceutical Sciences 62: 115-123.
Lipshultz, S. E., S. D. Colan, R. D. Gelber, A. R. Perez-Atayde, S. E. Sallan and S. P. Sanders (1991). “Late Cardiac Effects of Doxorubicin Therapy for Acute Lymphoblastic Leukemia in Childhood.” New England Journal of Medicine 324(12): 808-815.
Mallick, S. and J. S. Choi (2014). “Liposomes: Versatile and Biocompatible Nanovesicles for Efficient Biomolecules Delivery.” Journal of Nanoscience and Nanotechnology 14(1): 755-765.
Manconi, M., D. Valenti, C. Sinico, F. Lai, G. Loy and A. M. Fadda (2003). “Niosomes as carriers for tretinoin: II. Influence of vesicular incorporation on tretinoin photostability.” International Journal of Pharmaceutics 260(2): 261-272.
Manosroi, A., P. Khanrin, W. Lohcharoenkal, R. G. Werner, F. Götz, W. Manosroi and J. Manosroi (2010). “Transdermal absorption enhancement through rat skin of gallidermin loaded in niosomes.” International Journal of Pharmaceutics 392(1): 304-310.
Mashal, M., N. Attia, G. Puras, G. Martinez-Navarrete, E. Fernández and J. L. Pedraz (2017). “Retinal gene delivery enhancement by lycopene incorporation into cationic niosomes based on DOTMA and polysorbate 60.” Journal of Controlled Release 254: 55-64.
Moghassemi, S. and A. Hadjizadeh (2014). “Nano-niosomes as nanoscale drug delivery systems: An illustrated review.” Journal of Controlled Release 185: 22-36.
Mokhtar, M., O. A. Sammour, M. A. Hammad and N. A. Megrab (2008). “Effect of some formulation parameters on flurbiprofen encapsulation and release rates of niosomes prepared from proniosomes.” International Journal of Pharmaceutics 361(1): 104-111.
Nagarajan, R. (2002). “Molecular Packing Parameter and Surfactant Self-Assembly: The Neglected Role of the Surfactant Tail.” Langmuir 18(1): 31-38.
Ojeda, E., G. Puras, M. Agirre, J. Zarate, S. Grijalvo, R. Eritja, G. Martinez-Navarrete, C. Soto-Sánchez, A. Diaz-Tahoces, M. Aviles-Trigueros, E. Fernández and J. L. Pedraz (2016). “The influence of the polar head-group of synthetic cationic lipids on the transfection efficiency mediated by niosomes in rat retina and brain.” Biomaterials 77: 267-279.
Paré, C. and M. Lafleur (2001). “Formation of Liquid Ordered Lamellar Phases in the Palmitic Acid/Cholesterol System.” Langmuir 17(18): 5587-5594.
Soliman, S. M., N. S. Abdelmalak, O. N. El-Gazayerly and N. Abdelaziz (2016). “Novel non-ionic surfactant proniosomes for transdermal delivery of lacidipine: optimization using 2(3) factorial design and in vivo evaluation in rabbits.” Drug Deliv 23(5): 1608-1622.
Tagami, T., M. J. Ernsting and S.-D. Li (2011). “Efficient tumor regression by a single and low dose treatment with a novel and enhanced formulation of thermosensitive liposomal doxorubicin.” Journal of Controlled Release 152(2): 303-309.
Tagami, T., J. P. May, M. J. Ernsting and S.-D. Li (2012). “A thermosensitive liposome prepared with a Cu2+ gradient demonstrates improved pharmacokinetics, drug delivery and antitumor efficacy.” Journal of Controlled Release 161(1): 142-149.
Taymouri, S. and J. Varshosaz (2016). “Effect of different types of surfactants on the physical properties and stability of carvedilol nano-niosomes.” Advanced biomedical research 5: 48-48.
Uchegbu, I. F. (1995). “Distribution, metabolism and tumoricidal activity of doxorubicin administered in sorbitan monostearate (Span 60) niosomes in the mouse.” Pharmaceutical Research 12(7): 1019-1024.
Uchegbu, I. F., J. A. Double, L. R. Kelland, J. A. Turton and A. T. Florence (1996). “The activity of doxorubicin niosomes against an ovarian cancer cell line and three in vivo mouse tumour models.” J Drug Target 3(5): 399-409.
Waddad, A. Y., S. Abbad, F. Yu, W. L. L. Munyendo, J. Wang, H. Lv and J. Zhou (2013). “Formulation, characterization and pharmacokinetics of Morin hydrate niosomes prepared from various non-ionic surfactants.” International Journal of Pharmaceutics 456(2): 446-458.
Wang, S., Y. Meng, C. Li, M. Qian and R. Huang (2015). “Receptor-Mediated Drug Delivery Systems Targeting to Glioma.” Nanomaterials (Basel, Switzerland) 6(1): 3.
Yadav (2010). “Proniosomal Gel: A provesicular approach for transdermal drug delivery.” Der Pharmacia Lettre 2(4).
Yuasa, M., Oyaizu, K., Yamaguchi, A., Hanyuu, Y., Kasahara, K., Komuro, M. (2008). Niosome Having Metal Porphyrin Complex Embedded Therein, Process For Producing The Same And Drug With The Use Thereof. U.S.
Zeng, W., Q. Li, T. Wan, C. Liu, W. Pan, Z. Wu, G. Zhang, J. Pan, M. Qin, Y. Lin, C. Wu and Y. Xu (2016). “Hyaluronic acid-coated niosomes facilitate tacrolimus ocular delivery: Mucoadhesion, precorneal retention, aqueous humor pharmacokinetics, and transcorneal permeability.” Colloids and Surfaces B: Biointerfaces 141: 28-35.

1. A phospholipid-free small unilamellar vesicle (PFSUV) composition, wherein the composition comprises:
(a) a steroid; and
(b) a nonionic surfactant;
wherein the molar ratio of steroid:nonionic surfactant is between 3:1 to 5:1.

2. The composition of claim 1, wherein the nonionic surfactant is selected from the following: Tween-80™; Tween-85™; Tween-65™; Tween-60™; Tween-40™; Tween-20™; Span 60™; Span 80™; Span 85™; Span 65™; Span 40™; Span 20™; Pluronic F-88™; polysorbate 20; a Triton X-100™; Brij 78™; Brij 52™; Brij 30™; Brij 56™; Brij 58™; Brij 35™; Myrj 52™; sorbitan ester; a polyglycerol alkyl ether; a glucosyl dialkyl ether; and a polyoxyethylene alkyl ether.

3. The composition of claim 1, wherein the steroid is a sterol.

4. The composition of claim 3, wherein the sterol is selected from one or more of: cholesterol; campesterol; sitosterol; stigmasterol; and ergosterol.

5. The composition of claim 1, wherein the steroid is cholesterol and the nonionic surfactant is Tween-80™.

6. The composition of claim 1, wherein the mean diameter is below 100 nm when measured using dynamic light scattering.

7. The composition of claim 1, wherein the mean diameter is about 80 nm when measured using dynamic light scattering.

8. The composition of any-ne-f claim 1, wherein the composition further comprises an apolipoprotein component.

9. The composition of claim 8, wherein the apolipoprotein component is selected from one or more of: Apo A-1; Apo A-2; Apo A-4; Apo A-V or Apo A5; Apo B48; Apo B100; Apo C-I; Apo C-II; Apo C-III; Apo C-IV; Apo D; Apo E; Apo H; and Apo L.

10. The composition of claim 8, wherein the apolipoprotein component is integrated into the PFSUV membrane or the apolipoprotein component interacts with the surface of the PFSUV.

11. The composition of claim 1, wherein the composition further comprises a drug or imaging agent.

12. The composition of claim 11, wherein the drug or imaging agent is targeted to the liver.

13. The composition of claim 11, wherein the weight ratio of drug:lipid is about 1:4 to 1:40.

14. The composition of claim 11, wherein the weight ratio of drug:lipid is about 1:4 to 1:25.

15. The composition of claim 11, wherein the drug or imaging agent is selected from: doxorubicin; chloroquine; imiquimod; R848; curcumin; and sodium diatrizoate.

16. A method for treating a liver disease, the method comprising administering an effective amount of a composition of claim 11 to a subject in need thereof.

17. The method of claim 16, wherein the liver disease is selected from one or more of the following: viral hepatitis; non-viral hepatitis; cholestatic liver disease; non-alcoholic steatohepatitis (NASH); non-alcoholic fatty liver disease (NAFLD); primary biliary cholangitis; liver fibrosis; biliary atresia; hemochromatosis; Wilson's disease; alpha-1 antitrypsin deficiency; hyperoxaluria; oxalosis with liver cirrhosis; hepatitis B; and liver cancer.

18. The method of claim 17, wherein the liver cancer is selected from: hepatocellular carcinoma, intrahepatic cholangiocarcinoma, and hepatoblastoma.

19.-29. (canceled)

30. A method of diagnosis or staging a liver disease, the method comprising administering to a subject a composition of claim 11.

31. The method of claim 30, wherein the drug or imaging agent is selected from: a diagnostic probe; a contrast agent; a radioactive agent; a radioactive dye; a radiopharmaceutical; a PET imaging agent; or an MRI imaging agent.

32.-33. (canceled)

34. A method for making PFSUVs, the method comprising: combining a steroid and a nonionic surfactant using a microfluidic mixer, wherein the molar ratio of steroid:nonionic surfactant is between 3:1 to 5:1.

35. The method of claim 34, wherein the microfluidic mixer is a staggered herringbone mixer (SHM).

36. The method of claim 34, wherein the microfluidic mixer, uses a two-channel microfluidic injection system.

37.-44. (canceled)
